SEQUENCE LISTING (1 sequence)

SEQ ID NO: 1 - protein, 4 aa, Homo sapiens
SFVG

(19) United States
(12) Patent Application Publication
(10) Pub. No.: US 2009/0012095 A1
(43) Pub. Date: Jan. 8, 2009

(54) HUMAN N-TYPE CALCIUM CHANNEL BLOCKERS

(75) Inventors: Robert Zelle, Stow, MA (US); Pravin Chaturvedi, Andover, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/111,573

(22) Filed: Apr. 29, 2008

Related U.S. Application Data
(62) Division of application No. 10/674,803, filed on Sep. 29, 2003, now abandoned.
(60) Provisional application No. 60/414,738, filed on Sep. 30, 2002.

(51) Int. Cl. A61K 31/495; A61K 31/445; A61P 25/00

(52) U.S. Cl. 514/252.12; 514/329

(57) ABSTRACT

This invention features a method for modulating human N-type calcium channel α_{1B+SFVG} subunit activity. The method includes administering to a subject in need thereof an effective amount of a compound of formula (I), (II), or (III):

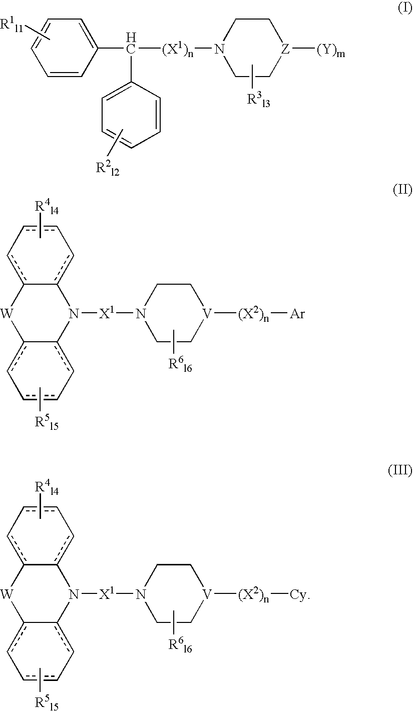

### CROSS REFERENCE TO RELATED APPLICATIONS

This application is a Divisional of U.S. patent application Ser. No. 10/674,803, which was filed on Sep. 29, 2003 and which claims the benefit of priority to U.S. Provisional Application No. 60/414,738, filed on Sep. 30, 2002. The contents of these applications are incorporated herein by reference in their entirety.

### BACKGROUND

Voltage gated calcium channels, also known as voltage dependent calcium channels are multisubunit membrane spanning proteins which permit controlled calcium influx from an extracellular environment into the interior of a cell. Several types of voltage gated calcium channel have been identified, such as N-type, P/Q-type, L-type and T-type channels.

A voltage gated calcium channel contains several subunits, including α₁, α₂, β, and γ subunits. Subtypes of the calcium channel subunits also are known. For instance, α₁ subtypes include α_{1A}, α_{1B}, α_{1C}, α_{1D}, α_{1E}, and α_{1S}. Each subunit may have one or more isoforms which result from alternative splicing of RNA in the formation of a completed messenger RNA which encodes the subunit. For example, at least four isoforms of the rat N-type α_{1B} subunit are known (see, e.g., Lin et al., Neuron 18:153-166, 1997).

A human N-type calcium channel isoform, α_{1B+SFVG}, has been identified, and is found to be involved in central nervous system signaling. See U.S. Pat. No. 6,353,091.

### SUMMARY

This invention relates to use of compounds in modulating human N-type calcium channel α_{1B+SFVG} subunit activity, and therefore, in treating a disorder associated with central nervous system signaling, such as stroke, pain (e.g., neuropathic pain), or traumatic brain injury.

In one aspect, this invention features a method for modulating (e.g., inhibiting or increasing) human N-type calcium channel α_{1B+SFVG} subunit activity. The method includes administering to a subject (e.g., a mammal, a human, a horse, a dog, or a cat) in need thereof an effective amount of a compound of formula (I), (II), or (III):

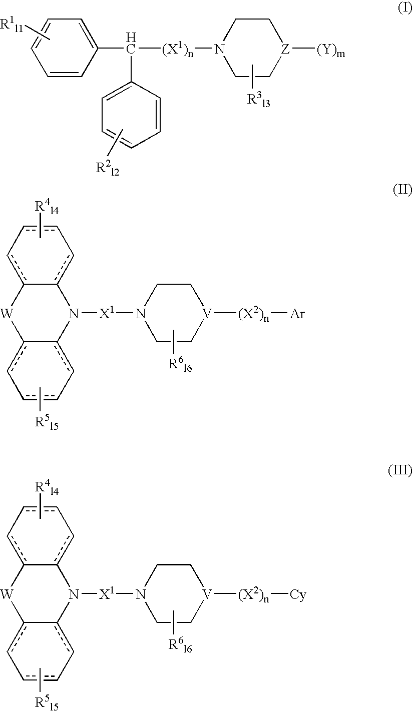

wherein
m is 0, 1 or 2, in which when m is 0, Z is O, when m is 1, Z is N, and when m is 2, Z is C; n is 0 or 1; each of X¹ and X², independently, is a linker; Y is H, OH, NH₂, or an organic moiety of 1-20C, optionally additionally containing 1-8 heteroatoms selected from the group consisting of N, P, O, S and halo; V is N or CH; W is O, S, NR or CR₂, in which R is H or alkyl (1-6C); Ar represents one or two substituted or unsubstituted aromatic or heteroaromatic rings; Cy represents one or two substituted or unsubstituted aliphatic cyclic or heterocyclic moieties, or consists of one substituted or unsubstituted aliphatic cyclic or heterocyclic moiety and one substituted or unsubstituted aromatic or heteroaromatic moiety; each of I1 and I2, independently, is 0, 1, 2, 3, 4, or 5; I3 is 0 or 1; each of I4 and I5, independently, is 0, 1, 2, 3, or 4; I6 is 0 or 1; each of R¹, R² and R³, independently, is alkyl (C1-C6), aryl (C6-C10), or arylalkyl (C7-C16) optionally containing 1-4 heteroatoms selected from the group consisting of halo, N, P, O, and S, or each of R¹ and R² may independently be halo, COOR, CONR₂, CF₃, CN or NO₂, in which R is H, lower alkyl (1-4C), or alkyl (1-6C); each of R⁴, R⁵ and R⁶, independently, is alkyl (1-6C), aryl (6-10C), or arylalkyl (7-16C) optionally containing 1-4 heteroatoms selected from the group consisting of halo, N, P, O, and S, or may independently be halo, OR, SR, NR₂, OOCR, NROCR, COR, COOR, CONR₂, CF₃, CN or NO₂, wherein R is H or alkyl (1-6C), and the dotted lines represent optional π-bonds; or compounds of formulae (II) or (III) where (X²)ₙ—Ar or (X²)ₙ-Cy is replaced by alkyl (1-6C); with the proviso that Y is not a tropolone, a coumarin, or an antioxidant containing an aromatic group, and with the further proviso that if I3 is 0, and either I1 and I2 is 0 or 1 and if R¹ and/or R² represent F in the para position, Z cannot be N or C.

In another aspect, this invention relates to a method for modulating (e.g., inhibiting or increasing) human N-type calcium channel α_{1B+SFVG} subunit activity, the method includes administering to a subject in need thereof an effective amount of a compound of formula (IV) or (V) or a pharmaceutically acceptable salt thereof:

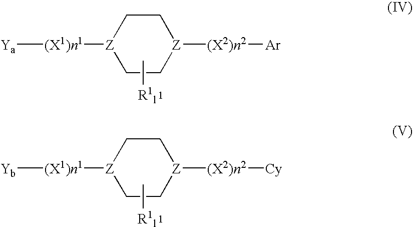

wherein, each Z is, independently, N or CH, at least one Z being N; n¹ is 1 and n² is 0 or 1; X¹ and X² are linkers; Ar represents one or two substituted or unsubstituted aromatic or heteroaromatic rings; Cy represents one or two substituted or unsubstituted aliphatic cyclic or heterocyclic rings, or consists of one substituted or unsubstituted aliphatic cyclic or heterocyclic ring and one substituted or unsubstituted aromatic or heteroaromatic ring. Each of Yₐ and Y_{b} is two substituted or unsubstituted aromatic or heteroaromatic rings, or two substituted or unsubstituted aliphatic cyclic or heterocyclic rings, or consists of one substituted or unsubstituted aliphatic cyclic or heterocyclic ring and one substituted or unsubstituted aromatic or heteroaromatic ring. In some embodiments, the rings cannot both be phenyl when both Ar includes a single phenyl ring and X¹ has less than 5 carbons. l¹ is 0 or 1; and R¹ is substituted or unsubstituted alkyl (C1-C6), substituted or unsubstituted aryl (C6-C10) or substituted or unsubstituted arylalkyl (C7-C16), each of which optionally further containing 1-4 heteroatoms selected from the group consisting of halo, N, P, O, and S; or is halo, OR, SR, NR₂, OOCR, NROCR, COR, COOR, CONR₂, CF₃, OCF₃, CN or NO₂, wherein R is H or alkyl (C1-C6). In some embodiments, formula (II) has at least one aromatic or heteroaromatic ring.

Other embodiments can include one or more of the following features.

Ar can represent one or two unsubstituted phenyl moieties.

n² can be 1 and X² can represent a linker which spaces Ar from Z at a distance of about 3-20 Å. In some embodiments, X² may contain at least one heteroatom selected from N and O. In some embodiments, Ar can represent one unsubstituted phenyl moiety and X² can represent —(CH₂)₁₋₈— or —(CH₂)₁₋₅—CH═CH—(CH₂)₀₋₀₃— or —NH(CH₂)₁₋₆—. In some embodiments, Ar can represent two phenyl moieties and X² can be of the formula —(CH₂)₀₋₆—CH.

Cy can represent one or two unsubstituted cyclohexyl moieties or an unsubstituted cyclohexyl moiety and an unsubstituted phenyl moiety.

n² can be 1 and X² can represent a linker which spaces Cy from Z at a distance of about 3-20 Å. In some embodiments, X² may contain at least one heteroatom selected from N and O. In some embodiments, Cy can be a cyclohexyl moiety, and X² can represent —(CH₂)₁₋₈— or —(CH₂)₁₋₅—CH═CH—(CH₂)₀₋₀₃— or —NH(CH₂)₁₋₆—. In some embodiments, Cy can represent two cyclohexyl moieties or a cyclohexyl moiety and a phenyl moiety. X can be —(CH₂)₀₋₆—CH. l¹ can be 0.

X¹ can represent a linker which spaces the Y^{a} and Y^{b} from N at a distance of about 3-20 Å. In some embodiments, X¹ may contains at least one heteroatom selected from O and N. X¹ can represent CH(CH₂)₀₋₉ or —CH(CH₂)₁₋₆CO.

In a further aspect, this invention relates to a method for modulating (e.g., inhibiting or increasing) human N-type calcium channel α_{1B+SFVG} subunit activity, the method includes administering to a subject in need thereof an effective amount of a compound of formula (VI) or a pharmaceutically acceptable salt thereof:

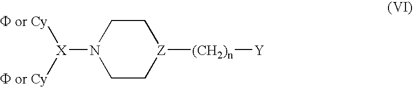

wherein, Cy represents cyclohexyl; Y is CH═CHΦ, CHΦ₂, Φ or Cy; X is divalent or trivalent straight-chain alkylene (C3-C10) or divalent or trivalent straight-chain 1-alkenylene (C3-C10) optionally substituted by oxo at the C adjacent N when n is 0 and Y is Φ₂CH; and is otherwise divalent or trivalent straight-chain alkylene (C5-C10) or divalent or trivalent straight-chain 1-alkenylene (C5-C10) optionally substituted by oxo at the C adjacent N; Z is N, NCO, CHNCOR¹ or CHNR¹, wherein R¹ is H or alkyl (C1-C6); and n is 0-5; wherein each Φ and Cy independently may optionally be substituted by alkyl (C1-C6) or by halo, CF₃, OCF₃, NO₂, NR₂, OR, SR, COR, COOR, CONR₂, NROCR or OOCR where R is H or alkyl (C1-4C), or two substituents may form a 5-7 membered ring. In some embodiments, the compounds having formula (VI) contain at least one aromatic moiety.

One subset of compounds include those having formula (VII).

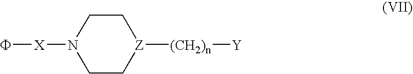

wherein X, Y, Z and n are as defined as above for formula (VI), and each Φ may optionally be substituted as set forth above.

Embodiments can include one or more of the following features.

Y can be CH═CHΦ.

In some embodiments, X can be CH(CH₂)ₘCO or CH(CH₂)ₘ₊₁ wherein m is 4-10, (e.g., 4, 5, 6, 7, 8, 9, 10). Z can be N and n can be 1-3.

Y can be Cy.

In some embodiments, X can be CH(CH₂)ₘCO or CH(CH₂)ₘ₊₁ wherein m is 4-10, (e.g., 4, 5, 6, 7, 8, 9, 10). Z can be N and n can be 1-3. 11. Z can be CH₂NH and n can be 1.

Y can be Φ₂CH.

In some embodiments, X can be CH(CH₂)ₗCO or CH(CH₂)ₗ₊₁, in which l is 1-10 (e.g., 1, 2, 3, 4, 5, 6, 7, 8, 9, 10). Z can be N.

n can be 0 or 1 and Y can be Φ.

In some embodiments, X can be CH(CH₂)ₘCO or CH(CH₂)ₘ₊₁ wherein m is 4-10, (e.g., 4, 5, 6, 7, 8, 9, 10).

The compound can be:

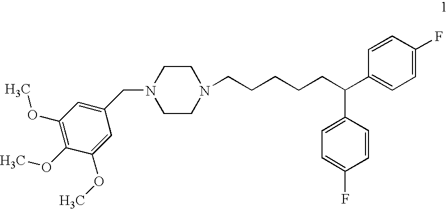

The compound can be:

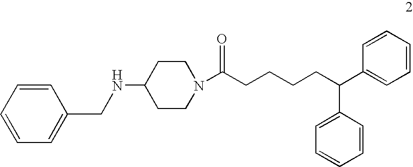

In one aspect, this invention relates to a method for modulating (e.g., inhibiting or increasing) human N-type calcium channel α_{1B+SFVG} subunit activity, the method includes administering to a subject in need thereof an effective amount of a compound of formula (VIII) or a pharmaceutically acceptable salt thereof:

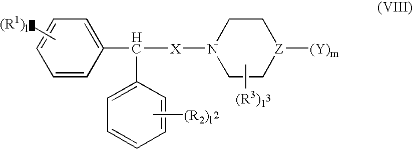

wherein m is 0, 1 or 2; wherein when m is 0, Z is O, when m is 1, Z is N, and when m is 2, Z is C. Y is H, OH, NH₂, or an organic moiety of C1-C20, optionally additionally containing 1-8 heteroatoms selected from the group consisting of N, P, O, S and halo; each l¹ and l² is independently 0-5; l³ is 0 or 1; each of R¹, R² and R³ is independently alkyl (C1-C6), aryl (C6-C10) or arylalkyl (C7-C16) optionally containing 1-4 heteroatoms selected from the group consisting of halo, N, P, O, and S or each of R¹ and R² may independently be halo, COOR, CONR₂, CF₃, CN or NO₂, wherein R is H or lower alkyl (C1-C4) or alkyl (C1-C6); n is 0 or 1; and X is a linker. In some embodiments, Y cannot be a tropolone, a coumarin, or an antioxidant containing an aromatic group. In some embodiments, if l³ is 0, neither R¹ nor R² can be F in the para position. In some embodiments, at least one of R¹, R² and R³ is a halo substituent.

Other embodiments may include one or more of the following features.

One subset of compounds include those having formula (IX):

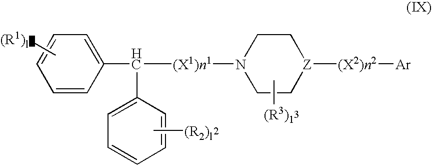

wherein Z is N or CH; wherein each of n¹ and n² is independently 0 or 1;
X¹ and X² are linkers; and Ar represents one or two substituted or unsubstituted aromatic or heteroaromatic rings.

Ar can represent one or two unsubstituted phenyl moieties.

n² can be 1 and X² can represent a linker which spaces Ar from Z at a distance of about 3-20 Å. In some embodiments, X² may contain at least one heteroatom selected from N and O. X² may represent —(CH₂)₁₋₈— or —(CH₂)₁₋₅—CH═CH—(CHCH₂)₀₋₃—. In some embodiments, Ar may represent two phenyl moieties and n² can be 1 and X² can be of the formula —(CH₂)₀₋₆—CH.

l³ can be 0.

l¹ and l² can be 0.

n¹ can be 1 and X¹ can represent a linker which spaces the benzhydril moiety from N at a distance of about 3-20 Å. In some embodiments, X¹ may contain at least one heteroatom selected from N and O. In some embodiments, X¹ may represent —(CH₂)₁₋₈— or —(CH₂)₁₋₅—CH═CH—(CHCH₂)₀₋₃—.

One subset of compounds include those having formula (X):

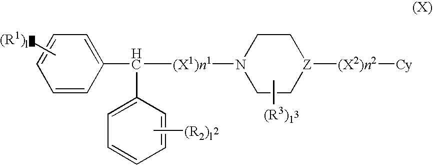

wherein Z is N or CH; wherein each of n¹ and n² is independently 0 or 1; X¹ and X² are linkers; and Cy represents one or two substituted or unsubstituted aliphatic cyclic or heterocyclic moieties or consists of one substituted or unsubstituted aliphatic cyclic or heterocyclic moiety and one substituted or unsubstituted aromatic or heteroaromatic moiety.

n² can be 1 and X² can represent a linker which spaces Cy from Z at a distance of about 3-20 Å. In some embodiments, X² may contain at least one heteroatom selected from N and O. In some embodiments, X² may represent —(CH₂)₁₋₈— or —(CH₂)₁₋₅—CH═CH—(CHCH₂)₀₋₃—.

l³ can be 0.

l¹ and l² can be 0.

n¹ can be 1 and X¹ can represent a linker which spaces the benzhydril moiety from N at a distance of about 3-20 A. In some embodiments, X¹ may contain at least one heteroatom selected from N and O. In some embodiments, X¹ may represent —(CH₂)₁₋₈— or —(CH₂)₁₋₅—CH═CH—(CHCH₂)₀₋₃—.

In another aspect, this invention relates to a method for modulating (e.g., inhibiting or increasing) human N-type calcium channel α_{1B+SFVG} subunit activity, the method includes administering to a subject in need thereof an effective amount of a compound having a straight backbone carbon chain of C8-C16, optionally substituted with 1-15 alkyl groups (C1-C6); said chain optionally functionalized at one terminus with halo, —OR, SR, NR₂, —OOCR, —NROCR wherein R is alkyl (C1-C6), or phosphate or pyrophosphate, or functionalized wherein a terminal carbon is optionally in the form of —COOR, —CONR₂ or COR wherein R is alkyl (C1-C16); and wherein said chain may optionally contain 1-4 π bonds or the epoxides thereof, or a pharmaceutically acceptable salt thereof. In some embodiments, the compound is other than farnesol or geraniol.

In a further aspect, this invention relates to a method of making a human N-type calcium channel α_{1B+SFVG} modulating compound, the method includes synthesizing a compound of any of the formulae described herein, e.g., formula (I), formula (II), formula (III), formula (IV), formula (V), formula (VI), formula (VII), formula (VIII), formula (IX), or formula (X), and contacting the compound with a human N-type calcium channel α_{1B+SFVG} subtype. The method can further include measuring the modulation of calcium channel activity, in which the contacting is conducted in vitro. Synthesizing the compound includes any of the transformations, intermediates, or reagents delineated herein (e.g., including those incorporated by reference).

In one aspect, this invention relates to a storage medium which includes chemical structure information of a compound of any of the formulae described herein, e.g., formula (I), formula (II), formula (III), formula (IV), formula (V), formula (VI), formula (VII), formula (VIII), formula (IX), or formula (X), and calcium channel activity of a human N-type calcium channel α_{1B+SFVG} when in contact with the compound. Also within the scope of this invention is a method of evaluating information using the storage medium, e.g., in a computer evaluation or drug discovery or design process, comprising using, (e.g., displaying, analyzing, transferring, manipulating, calculating) data from the storage medium with computer software to display or analyze the information in a process for determining the usefulness of the compound as a therapeutic drug candidate.

In another aspect, this invention relates to a method of measuring the modulation activity of a compound of any of the formulae described herein, e.g., formula (I), formula (II), formula (III), formula (IV), formula (V), formula (VI), formula (VII), formula (VIII), formula (IX), or formula (X), against a human N-type calcium channel α_{1B+SFVG} subunit, the method includes providing a compound e.g., formula (I), formula (II), formula (III), formula (IV), formula (V), formula (VI), formula (VII), formula (VIII), formula (IX), or formula (X), and contacting the compound with a human N-type calcium channel α_{1B+SFVG} subunit.

In a further aspect, the invention relates to a method for modulating (e.g., inhibiting or increasing) human N-type calcium channel α_{1B+SFVG} subunit activity in a cell that includes contacting a compound (or pharmaceutically acceptable derivative thereof) of any of the formulae herein (or a composition including the compound or derivative thereof) with the cell. The contacting can be in vitro or in vivo, and can include administration of the compound of any of the formulae herein to a vessel (e.g., petri dish, test tube, etc.) or to a subject (e.g., mammal, human, dog, cat, horse, monkey, rat, mouse, sheep) having the cell therein.

The methods include administering to the subject (including a subject identified as in need of such treatment, that is identified as in need of human N-type calcium channel α_{1B+SFVG} modulation) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the term “alkyl” refers to a hydrocarbon chain that may be a straight chain or branched chain, containing the indicated number of carbon atoms. For example, C₁˜C₅ indicates that the group may have from 1 to 5 (inclusive) carbon atoms in it.

The term “aryl” or “aromatic ring” refers to an aromatic 5-8 membered monocyclic or 8-12 membered bicyclic, or tricyclic hydrocarbon ring system, wherein any ring atom capable of substitution can be substituted by a substituent. Examples of aryl moieties include, but are not limited to, phenyl, naphthyl, anthracenyl, perylenyl, and pyrenyl. The term “Φ” refers to substituted or unsubstituted phenyl.

The term “arylalkyl” refers to a moiety in which an alkyl hydrogen atom is replaced by an aryl group.

The term “linker” refers to a chemical group containing 1-10 carbon atoms which may be saturated or unsaturated and may contain a ring. The linker may also contain one or two heteroatoms selected from N, O and S, and may be substituted.

As used herein, the term “halo” or “halogen” refers to any radical of fluorine, chlorine, bromine or iodine.

The term “cycloalkyl” or “aliphatic cyclic ring” as used herein includes saturated cyclic, bicyclic, tricyclic, or polycyclic hydrocarbon groups having 3 to 12 carbons. Any ring atom can be substituted. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” or “aliphatic hetrocyclic ring” refers to a nonaromatic 3-10-membered monocyclic, 8-12 membered bicyclic, or 11-14 membered tricyclic ring system having 1-3 heteroatoms if monocyclic, 1-6 heteroatoms if bicyclic, or 19 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “heteroaryl” or “heteroaromatic ring system” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom capable of substitution can be substituted. Examples of heterocyclyl include, but are not limited to, pyridyl, furyl or furanyl, imidazolyl, pyrimidinyl, thiophenyl or thienyl, quinolinyl, indolyl and the like.

The term “substituted” refers to a group, e.g., an alkyl, cycloalkyl, heterocyclyl, aryl, or heteroaryl group, having one or more of its hydrogen atoms (e.g. 1 hydrogen atom, 2 hydrogen atoms, 3 hydrogen atoms, 4 hydrogen atoms, 5 hydrogen atoms, 6 hydrogen atoms, 7 hydrogen atoms, 8 hydrogen atoms, 9 hydrogen atoms, 10 hydrogen atoms, 11 hydrogen atoms, 12 hydrogen atoms) replaced by one or more “substituents.” Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, for groups having two or more substituents, the substituents may be the same or different. In another aspect, a substituent may itself be substituted with any one of the above substituents.

The term “human N-type calcium channel α_{1B+SFVG} subunit” (“hα_{1B+SFVG} subunit”) refers to any human N-type calcium channel α_{1B} subunit clone that contains the SFVG sequence set forth in SEQ ID NO: 1: Ser-Phe-Val-Gly. The nucleotide sequence and the amino acid sequence of the hα_{1B+SFVG} subunit have been described in U.S. Pat. No. 6,353,091. The term also includes functionally equivalent variants, useful analogs and fragments of the nucleic acids and polypeptides of the hα_{1B+SFVG} subunit.

As used herein “human N-type calcium channel α_{1B+SFVG} subunit activity” (“hα_{1B+SFVG} subunit activity”) refers to an ability of a compound to modulate voltage regulated calcium influx. A compound which inhibits hα_{1B+SFVG} subunit activity (an antagonist) is one that inhibits voltage regulated calcium influx via this calcium channel and a compound which increases hα_{1B+SFVG} subunit activity (an agonist) is one that increases voltage regulated calcium influx via this calcium channel.

In another aspect, this invention features a method for treating a disorder associated with central nervous system (CNS) signaling. The method includes administering to a subject in need thereof an effective amount of a compound of formula (I), (II), or (III) described above. The disorder can be stroke, pain (e.g., neuropathic pain), or traumatic brain injury. Other examples of CNS disorders include neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, and Jakob-Creutzfieldt disease; psychiatric disorders, e.g., depression, schizophrenic disorders, Korsakoff's psychosis, mania, anxiety disorders, or phobic disorders; learning or memory disorders, e.g., amnesia or age-related memory loss; and neurological disorders, e.g., migraine.

Also within the scope of this invention is a packaged product. The packaged product includes a container, one of the aforementioned compounds in the container, and a legend (e.g., a label or an insert) associated with the container and indicating administration of the compound for treating a disorder associated CNS signaling.

Other features, objects, and advantages of the invention will be apparent from the description and from the claims.

### DETAILED DESCRIPTION

The compounds of formula (I)-(X) described above, useful in the methods of the invention, exert their desirable effects through their ability to modulate the activity of human N-type calcium channel α_{1B+SFVG} subunit.

The compounds of formula (I) are defined as shown in terms of the embodiments of their various substituents:

Z may be O, N, or C, where m has the appropriate value, i.e., O when m is 0, N when m is 1, or C when m is 2. When m is 2, one of the Y substituents is preferably H, OR, NR₂, wherein R is H, alkyl (C1-C6), or one Y may be itself alkyl (C1-C6). Preferred forms of Z are N and C, where one Y is H or OH.

Y is H, OH, or NH₂, or an organic moiety of 1-16C, optionally additionally containing 1-8 heteroatoms selected from the group consisting of N, P, O, S and halo. Preferred forms of at least one Y include those that comprise an aromatic ring system, including fused ring systems and rings containing one or more heteroatoms. Particularly preferred forms of at least one Y are those which include phenyl moieties. The aromatic moieties included within Y may be substituted or unsubstituted; the “substituents” may include alkyl (C1-C6), halo, OR, SR, NR₂, COOR, or CONR₂, wherein each R is independently H, alkyl (1-6C), CN, CF₃, or NO₂. This set of moieties will be referred to herein as “1^{st} set substituents.” Of course, if Z is 0, Y is not present (m=0). Additional preferred embodiments of Y include: aminoindane, azulene, cyclohexane, cyclohexanol, hexahydroazepin, indane, indene, indazole, indole, indolazine, morpholine, phenothiazine, phenoxazine, piperidine, pyrrole, pyridine, pyrimidine, thionaphthene, thiomorpholine, thiazine, and thiazole. When m is 2, the two Y groups may be the same or different, and preferred forms are those set forth above. Particularly preferred, however, are embodiments where, when m is 2 and Z is C, one Y is selected from the foregoing list and the other Y is H or OH.

R³ may be alkyl (C₁₋₆C), aryl (C6-10C), or arylalkyl (C7-16C) optionally containing 1-4 heteroatoms selected from the group consisting of N, P, O, S, and halo; preferred embodiments of R³ include methyl. Typically, I³ is 0 or 1.

As n may be 0 or 1, X may be present or not. X is a suitable linker containing 1-10C which may be saturated or unsaturated and may contain a ring. The linker may also contain one or two heteroatoms selected from N, O and S, and may be substituted with the “1^{st} set substituents” listed above. Preferred embodiments of X include —(CH₂)ₚ—, wherein p is an integer of 1-10, preferably 1-6, and more preferably 1-4 or 1-2.

R¹ and R² may independently be alkyl (C1-C6), aryl (C6-C10), or arylalkyl (C7-C16) optionally containing 1-4 heteroatoms and optionally containing any of the “1^{st} set substituents” set forth above, or R¹ and R² may themselves independently be said substituents; I1 and I2 are each independently 0-5, but preferably 0-3. Preferred embodiments of I1 and I2 include 1, where the substituent is in the para position (1p) or 3, where the substituents are in the two ortho positions and the para position (3o,p) or 2, where the substituents are in the meta positions (2m). Preferred forms of R¹ and R² include phenyl, phenylalkyl, halo, CF₃, amino, and alkyl.

The compounds of formulae (II) or (III) are defined as shown in terms of the embodiments of their various substituents:

W may be O, S, NR or CR₂; preferably each R is H. More preferably, W is S.

V may be N or CH.

Preferably, each of R⁴ and R⁵ is independently alkyl (C1-C6), arylalkyl (C7-C16), halo, OR, SR, NR₂, OOCR, NROCR, COR, COOR or CONR₂, wherein each R is independently H or alkyl (C1-C6), or may be CN, CF₃ or NO₂ (“2^{nd} set substituents”). Preferred embodiments of 14 and 15 include those 1 (where one substituent is ortho or meta to W and 2), where two substituents are in the positions meta and para to W. Especially preferred forms of R⁴ and R⁵ include phenyl, phenylalkyl, F, Cl, Br, I, CF₃, OR, NR₂ and alkyl. Particularly preferred are F, OMe, NH₂, NMe₂, NHOAc, CONH₂, Br, COOEt, and COOMe.

R⁶ may be alkyl (C1-C6), aryl (C6-C10), or arylalkyl (C7-C16) optionally containing 1-4 heteroatoms selected from the group consisting of N, P, O, S, and halo; preferred embodiments of R⁶ include methyl. R⁶ may also include halo, OR, SR, NR₂, OOCR, NROCR, COR, COOR or CONR₂, wherein each R is independently H, alkyl (C1-C6), or may be CN, CF₃ or NO₂. Typically, I6 is 0 or 1, preferably 0.

As n may be 0 or 1, X² may be present or not. X¹ and X² are suitable linkers containing 1-10C which may be saturated or unsaturated and may contain a ring. The linker may also contain one or two heteroatoms selected from N, O and S and may be substituted with the “2^{nd} set substituents” listed above. Preferred embodiments of X¹ and X² include —(CH₂)ₚ— wherein p is an integer of 1-10, preferably 1-6, —(CH₂)_{q}—CO— or —CO(CH₂)_{q}—, where q is an integer of 1-9, and —(CH₂)ₙ—CH═CH—, where s is an integer of 0-4. Also preferred particularly for X² is —NH(CH₂)ₜ— or —NHCO(CH₂)ₜ—, where t is an integer of 1-8, when Z is CH.

Thus, formulae (II) and (III) are similar, except that compounds of formula (II) contain aromatic substituents linked to the heterocyclic 6-membered ring and those of (III) contain aliphatic cyclic or heterocyclic moieties. In each case, preferably when X² is present, X² represents a linker which spaces the Ar or Cy moiety from V at a distance of 3-20 Å, and may contain at least one heteroatom which is nitrogen or oxygen. Included in such linkers are amines and carbonyl functionalities, including amides. The linker may also be unsaturated or may be an alkylene group. Typically, X² is —(CH₂)₁₋₁₀— or —CO(CH₂)₁₋₉—, or —(CH₂)₁₋₅—CH═CH—(CH₂)₀₋₃—. Similarly, X¹ spaces the constrained fused ring system from the nitrogen of the heterocyclic ring at a distance of about 3-20 Å.

For X², when there are two aromatic or heterocyclic or other cyclic moieties, X² must accommodate this and a typical embodiment is —(CH₂)₀₋₉—CH—. X² may also contain a π-bond, e.g., —(CH₂)₀₋₅—CH═C—, for such accommodation.

In preferred forms of formulae (II) and (III), X¹ is —(CH₂)₁₋₅CO(CH₂)₀₋₃— or —(CH₂)₁₋₅NH(CH₂)₁₋₃—, or —(CH₂)₁₋₅CONH(CH₂)₁₋₃—.

Preferred embodiments for X² are similar except that in instances where Ar or Cy represent two rings, the two rings are coupled to CH or to a π-bonded carbon as the terminal portion of the linker X².

Although it is preferred that 14 and 15 are both 0, substitution by R⁴ and R⁵ in the constrained fused ring system is permitted as set forth in the description of the invention above. It is believed that halogenation of the compounds of the invention is helpful in modulating the in vivo half-life, and it may be advantageous to include halogen substituents as R⁴ and R⁵. In formulae (II) and (III), such substituents may also be included on Ar and Cy.

The compounds described above may be synthesized using conventional methods. See, e.g., U.S. Pat. Nos. 6,011,035; 6,267,945; 6,294,533; 6,310,059; 6,387,897; 6,492,375; U.S. Patent Application Publication Nos. 2001/0029258 A1 and 2003/0045530 and references cited therein, all incorporated by reference in their entirety. As can be appreciated by the skilled artisan, the aforementioned synthetic methods are not intended to comprise a comprehensive list of all means by which the compounds described in this application may be synthesized. Further methods will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The synthesized compound can be separated from a reaction mixture and further purified by a method such as column chromatography, high pressure liquid chromatography, or recrystallization.

As used herein, the compounds described above, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of one of the aforementioned compounds which, upon administration to a recipient, is capable of providing (directly or indirectly) the compound. Particularly favored derivatives and prodrugs are those that increase the bioavailability of the compounds described above when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. The compounds may contain one or more asymmetric centers and one or more double bonds. Thus, they can occur as racemates and racemic mixtures, single enantiomers, individual diastereomers, diastereomeric mixtures, and cis- or trans or E- or Z-double bond isomeric forms. All such isomeric forms of these compounds are contemplated. All crystal forms of the compounds described herein are expressly included in the present invention.

The aforementioned compounds may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Note that combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Also within the scope of this invention is a pharmaceutical composition that contains an effective amount of at least one of the compound described above and a pharmaceutically acceptable carrier. This invention covers a method of administering an effective amount of one or more compounds of this invention to a subject in need of inhibiting human N-type calcium channel α_{1B+SFVG} subunit activity. This invention also covers a method of administering an effective amount of one or more compounds described above to a subject in need of treating a disorder associated with central nervous system signaling, such as stroke, pain, e.g., neuropathic pain, and traumatic brain injury.

The term “treating” or “treated” refers to administering a compound described above to a subject with the purpose to cure, heal, alleviate, relieve, alter, remedy, ameliorate, improve, or affect a disease, the symptoms of the disease or the predisposition toward the disease. “An effective amount” refers to an amount of a compound which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/kg to about 15 mg/kg, alternatively about 0.1-1.0 mg/kg. Effective doses will also vary depending on route of administration, as well as the possibility of co-usage with other agents for treating a disorder associated with central nervous system signaling.

To practice the method of treating a disease, the compounds can be administered to a patient, for example, in order to treat a disorder described above. The compound can, for example, be administered in a pharmaceutically acceptable carrier such as physiological saline, in combination with other drugs, and/or together with appropriate excipients. The compound described herein can, for example, be administered by injection, intravenously, intraarterially, subdermally, intraperitoneally, intramuscularly, or subcutaneously; or orally, buccally, nasally, transmucosally, topically, in an ophthalmic preparation, by inhalation, by intracranial injection or infusion techniques, with a dosage ranging from about 0.5 to about 100 mg/kg of body weight, preferably dosages between 1 and 1000 mg/dose, every 4 to 120 hours, or according to the requirements of the particular drug. The methods herein contemplate administration of an effective amount of compound or compound composition to achieve the desired or stated effect. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

The term “pharmaceutically acceptable carrier” refers to a carrier (adjuvant or vehicle) that may be administered to a patient, together with a compound described above, and which does not destroy the pharmacological activity thereof and is nontoxic when administered in doses sufficient to deliver a therapeutic amount of the compound.

Pharmaceutically acceptable carriers that may be used in the pharmaceutical compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A sterile injectable composition (e.g., aqueous or oleaginous suspension) can be formulated according to techniques known in the art using suitable dispersing or wetting agents (such as, for example, Tween 80) and suspending agents.

The pharmaceutical compositions may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A suitable in vitro assay can be used to preliminarily evaluate a compound described above in modulating human N-type calcium channel α_{1B+SFVG} subunit activity. For example, in vitro assays, such as patch-clamp assays and assays employing calcium sensitive fluorescent compounds (e.g., fura-2) are used to determine the changes in hα_{1B+SFVG} subunit activities. In vivo screening can also be performed by following procedures well known in the art. See also, Lipscombe et al., “Human N-Type Calcium Channel Isoform”, U.S. Pat. No. 6,353,091 (Mar. 5, 2002), and references cited therein, all incorporated by reference in their entirety.

Numerous computer programs are available and suitable for rational drug design and the processes of computer modeling, model building, and computationally identifying, selecting and evaluating potential modulator compounds, e.g., inhibitors, in the methods described herein. These include, for example, QSC (WO 01/98457), FlexX, Autodock, Glide, Accelrys' Discovery Studio, or Sybyl. Potential modulator compounds can also be computationally designed “de novo” using such software packages as QSC (WO 01/98457), Accelrys' Discovery Studio, Sybyl, ISIS, ChemDraw, or Daylight. Compound deformation energy and electrostatic repulsion, can be evaluated using programs such as GAUSSIAN 92, AMBER, QUANTA/CHARMM, AND INSIGHT II/DISCOVER.

These computer evaluation and modeling techniques can be performed on any suitable hardware including for example, workstations available from Silicon Graphics, Sun Microsystems, and the like. These techniques, methods, hardware and software packages are representative and are not intended to be comprehensive listing. Other modeling techniques known in the art can also be employed in accordance with this invention. See for example, QSC (WO 01/98457), FlexX, Autodock, Glide, Accelrys' Discovery Studio, or Sybyl and software identified at various internet sites (e.g.,

netsci.org/Resources/Software/Modeling/CADD/ch.cam.ac.uk/SGTL/software.html
cmm.info.nih.gov/modeling/universal_software.html
dasher.wustl.edu/tinker/zeus.polsl.gliwice.pl/˜nikodem//linux4chemistry.html
nyu.edu/pages/mathmol/software.html
msi.umn.edu/user_support/software/MolecularModeling.html
us.expasy.org/sisweb.com/software/model.htm).

Generally, a computer will include one or more mass storage media or devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

### EXAMPLE

Representative compounds of the formulae herein are screened for activity against potassium channel targets in an assay essentially as described in J. Neurosci., Aug. 15, 2001, 21(16):5944-5951, W. Xu and D. Lipscombe, using transient expression and recording from Xenopus oocytes. The assay is performed using various calcium channels (e.g., N-type calcium channel α_{1B+SFVG}) whereby the modulation of the calcium channel is measured for each compound.

The following IC50 data were obtained for compounds 1 and 2, following the above-referenced protocols (see Table 1).

TABLE 1
IC₅₀ (μm)

| Compound | Oocytes | HEK298 cells |
|---|---|---|
| 1 | 7.13 | 0.19 |
| 2 | 4.16 | — |

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for modulating human N-type calcium channel α_{1B+SFVG} subunit activity, comprising administering to a subject in need thereof an effective amount of a compound of formula (II): 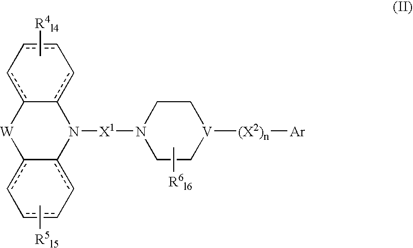
wherein,
n is 0 or 1;
each of X¹ and X², independently, is a linker;
V is N or CH;
W is O, S, NR or CR₂, in which R is H or alkyl (C1-C6);
Ar represents one or two substituted or unsubstituted aromatic or heteroaromatic rings;
each of I4 and I5, independently, is 0, 1, 2, 3, or 4;
I6 is 0 or 1;
each of R⁴, R⁵ and R⁶, independently, is alkyl (C1-C6), aryl (C6-C10) or arylalkyl (C7-C16) optionally containing 1-4 heteroatoms selected from the group consisting of halo, N, P, O, and S, or may independently be halo, OR, SR, NR₂, OOCR, NROCR, COR, COOR, CONR₂, CF₃, CN or NO₂, wherein R is H or alkyl (C1-C6), and the dotted lines represent optional π-bond).
